(19) United States
(12) Patent Application Publication
(10) Pub. No.: US 2026/0224133 A1
(43) Pub. Date: Aug. 6, 2026

(54) SPEECH AUDIOMETRY TEST METHOD USING VOICE RECOGNITION AND ASSOCIATED ELECTRONIC DEVICE

(71) Applicant: MY MEDICAL ASSISTANT, REIMS (FR)

(72) Inventors: Nicolas WALLAERT, REIMS (FR); Hadrien JEAN, BOLOGNA (IT)

(73) Assignee: MY MEDICAL ASSISTANT, REIMS (FR)

(21) Appl. No.: 18/868,938

(22) PCT Filed: May 24, 2023
(86) PCT No.: PCT/IB2023/055334

§ 371 Date: Nov. 25, 2024

(30) Foreign Application Priority Data
May 25, 2022 (FR) FR2205043

(51) Int. Cl. A61B 5/12; G10L 15/06; G10L 15/16; G10L 15/187

(52) U.S. Cl. CPC A61B 5/123; G10L 15/063; G10L 15/16; G10L 15/187; G10L 2015/0638

(57) ABSTRACT

A method of speech audiometry testing of a first patient includes first acoustic emission of the recording of a linguistic expression comprising at least one emission phoneme, first acoustic reception of a first vocal response from the first patient to the first acoustic emission comprising at least one response phoneme, determination, by an artificial neural network including an input and an output, on the basis of input data obtained from the first response, of a first output character string representative of at least one response phoneme, and comparison of the first character string with a second character string, representative of said at least one input phoneme.

The invention concerns a speech audiometry method.

The speech audiometry test procedures carried out by an audiologist make it possible to determine a patient's audio perception of linguistic expressions, particularly words.

These processes include:
The acoustic emission of a recording of a linguistic expression,
Acoustic reception and recognition of the response of a first patient, and
Comparison of the linguistic expression with the patient's response.

Recognition of the response is carried out by the audiologist or, more generally, by a person (in other words: human), which requires the mobilization of a person for the duration of the test.

To remedy this drawback, the invention relates to a method for speech audiometry testing of a first patient comprising the following steps:
First acoustic emission of a recording of a linguistic expression comprising at least one emission phoneme (The first patient then reproduces by speech what he has heard. In other words, he emits a first vocal response),
First acoustic reception of a first vocal response from the first patient to the first acoustic emission comprising at least one phoneme in the response,
Determination, by an artificial neural network comprising an input and an output, from input data obtained from the first response, of a first output character string representative of at least one response phoneme,
Comparison of the first character string with a second character string, representative of said at least one input phoneme (in other words: representative of said at least one output phoneme),

The artificial neural network being trained, prior to the determination step, by implementing (or repeating) the following training steps (and the test method may comprise these steps):
Second acoustic emission of expression (The second patient mentioned below then reproduces in speech what he has heard. In other words, he emits a vocal response),
Second acoustic reception of a second vocal response from a second patient to the second acoustic transmission, the second response comprising at least one training phoneme (the second response is then recorded in memory, for example, in an audio file),
Listening to the second answer by a human (i.e., the audio file),
Reception of a third character string representing at least the training phoneme,
Supervised training of the artificial neural network based on the second input response labelled with the third character string (i.e., the training of the neural network tends to result in the neural network outputting the third character string when the second response is received as input).

In this way, the neural network can automate the acquisition of the patient's response. Training the neural network from expression enables:
Avoidance of over-interpretation, as with conventional speech recognition neural networks (i.e., conventional neural networks look for the closest word in the language, even if the patient has not answered that word).
The network is trained to recognize a patient's response, even when the word answered by the patient is not a word in the language. Since the network is trained during speech audiometry tests, it receives words that are not words in the language (because the words emitted are meaningless, or because the patient responds with an error).

The steps in the above process can be repeated in order to assess the hearing of the first patient, for example for different input words (in the expression) for which the neural network has been trained. The intensity of the acoustic expression can be varied during this repetition to estimate the patient's speech intelligibility thresholds.

In one embodiment, the expression consists of one word (or several isolated words), or one word (or several words) preceded by an isolated article. Alternatively, the expression consists of more than one word (or more than two words) or one or more sentences.

The training step is preferably repeated with at least several different second patients. According to one embodiment, the training step is repeated (for example, at least 10 000 times) for, as input, less than 300 different expressions (and for example, more than 50 words) constituting lists, for different second patients. For example, these lists are Lafon's cochlear lists or Fournier's dissyllabic lists.

Alternatively, the training step can be repeated for a larger number of words.

Preferably, the training steps (or process steps) are repeated (for example, at least 10 000 times) for a series of different expressions and/or for different second patients.

The series of different expressions consists of (or comprises) less than 2000 different expressions, for example, less than 300 different expressions (the series of different expressions comprising at least 10 for example).

For example, the steps of the audiometric test procedure are repeated for part of the series of expressions.

According to an embodiment, all or some of the different second patients are hard of hearing (or the second patient is hard of hearing). A patient is hard of hearing if, for example, at least one of his ears has an average hearing loss of more than 20 (or 41 or 71) decibels on at least one audiometric frequency, for example, equal to 500 hertz, 1000 hertz, 2000 hertz or 4000 hertz.

The input to the neural network can be made up of several expressions. It is more efficient to allow the neural network to work on several expressions at the same time.

For example, the neural network comprises a first part, comprising a first series of layers of the neural network, capable of producing a vector encoding the input data, and a second part, comprising at least one layer of the neural network, capable of producing the first output character string from the vector, the first part being pre-trained (and the method may comprise this pre-training step), prior to the training steps, from more than 10000, at least, different input expressions (or words) (and from at most 10000000 different expressions or words).

In one embodiment, at least a portion of the first part (e.g., connected to the input) is fixed in weight (i.e., connections between layers) during the training step. The rest of the neural network, apart from the said at least one portion, is modified during training. Alternatively, the entire first part can be trained.

The first part is, for example, the neural network described in the article: Alexei Baevski, Yuhao Zhou, Abdelrahman-Mohamed, Michael Auli: “wav2vec 2.0: A Framework for Self-Supervised Learning of Speech Representations” NeurIPS 2020.

According to an embodiment, in this first part, the weights, of the convolutional encoder (noted f) of the item which produces a latent representation of the input are fixed during the training step. The rest of the first part, particularly the transformer (noted g), is modified during training.

Other neural networks than the one presented in this article are of course possible.

The data is, for example, an audio file (or part of an audio file) comprising the audio recording of the first response (for example in a format known as “wav”, in English for “Waveform Audio File Format” translated into French by the expression format audio de forme d'onde).

In one embodiment, the first part is pre-trained from a truncated input (for example, certain parts of the audio recording have been removed).

Alternatively, the first part is trained using labelled data.

So, following pre-training, the first part is able to encode an input audio file (or audio data) into a vector comprising a context representation.

Alternatively, the data is an image file, for example a spectrogram obtained from the first voice response. Such an approach is presented for general speech recognition in the following article:

Zhang, Wei, et al. “Towards end-to-end speech recognition with deep multipath convolutional neural networks.” International Conference on Intelligent Robotics and Applications. Springer, Cham, 2019.

For example, the training stages include the following step:
Keyboard input of the third string of characters by the human, following the listening stage.

Input can be via a keyboard or any other type of human-machine interface.

For example, the electronic device stores the voice recording in memory.

The first acoustic output of the recording of a linguistic expression can, for example, be produced by headphones or a loudspeaker.

The second acoustic emission of the recording of a linguistic expression can, for example, also be produced by headphones or a loudspeaker.

The first reception of a first response and/or the second reception of the second response is carried out, for example, by a microphone.

The artificial neural network is, for example, a convolutional network. The first part may include a transform. In the case where the first part is the neural network presented in the Alexei Baevski et al. article above, the second part can consist of a single output layer comprising all the phonemes contained in the lists (the output vector indicates which phonemes were in the input).

The artificial neural network can be implemented by the central processing unit, which can have the architecture of a computer, a microprocessor and a microcontroller.

By comparing the first character string with the second character string, the hearing ability of the first patient can be determined in a conventional way.

The second response can be heard through headphones.

Each of the above headphones can be replaced by a loudspeaker.

The process is implemented, for example, by an electronic device.

The invention therefore also relates to an electronic audiometric testing device configured to implement the steps of the method according to the invention.

The invention also relates to a computer program comprising instructions, executable by a microprocessor or microcontroller, for implementing the method according to the invention, when the computer program is executed by a microprocessor or microcontroller.

The characteristics and advantages of the electronic device and the computer program are identical to those of the process, which is why they are not repeated here.

According to a variant, the invention relates to a method for speech audiometry of a first patient comprising the following steps:
First acoustic emission of a recording of a linguistic expression comprising at least one emission phoneme (The first patient then reproduces by speech what he has heard. In other words, he emits a vocal response),
First acoustic reception of a first vocal response from the first patient to the first acoustic emission comprising at least one response phoneme,
Determination, by an artificial neural network comprising an input and an output, on the basis of input data obtained from the first response, of a first output character string representative of a comparison of said at least one response phoneme with the at least one emission phoneme,

The artificial neural network being trained, prior to the determination step, by implementing (or repeating) the following training steps (and the test method may comprise these steps):
Second acoustic emission of the expression (The second patient mentioned below then reproduces in speech what he has heard. In other words, he emits a second vocal response),
Second acoustic reception of a second vocal response from a second patient to the second acoustic transmission, the second response comprising at least one training phoneme (the second response is then recorded in memory, for example, in an audio file),
Reception of a third character string representing a comparison of the at least one transmission phoneme with at least one training phoneme,
Supervised training of the artificial neural network based on the second input response labelled with the third character string (i.e., the training of the neural network tends to result in the neural network outputting the third character string when the second response is received as input).

The hearing ability of the first patient can be determined conventionally from the first character string.

Compared to the speech audiometry test method, the speech audiometry method may have the advantage of not requiring reception of the training phoneme, which may avoid, for example, a typing during training following the second response.

The advantages and characteristics of the speech audiometry method are identical to those of the above speech audiometry test method (without needing to be repeated here), apart from the listening and comparison step.

The invention therefore also relates to an electronic audiometric device configured to implement the steps of the audiometry method according to the invention.

The invention also relates to a computer program comprising instructions, executable by a microprocessor or microcontroller, for implementing the speech audiometry method according to the invention, when the computer program is executed by a microprocessor or microcontroller.

The characteristics and advantages of the electronic audiometric device and the computer program are identical to those of the audiometry process, which is why they are not repeated here.

An element such as an electronic device, central processing unit or other element is “configured to” perform a step or operation by the fact that the element has means for (in other words, is “shaped to” or “adapted to”) perform the step or operation. These are preferably electronic means, for example a computer program, data in memory and/or specialized electronic circuits.

Where a step or operation is carried out or implemented by such an element, this generally implies that the element includes means for (in other words “is shaped to” or “is adapted to”) carry out the step or operation. This also includes, for example, electronic means, such as a computer program, stored data and/or specialized electronic circuits.

Other features and advantages of the present invention will become clearer on reading the following detailed description comprising modes of carrying out the invention given by way of non-limiting examples and illustrated by the appended drawings, in which:

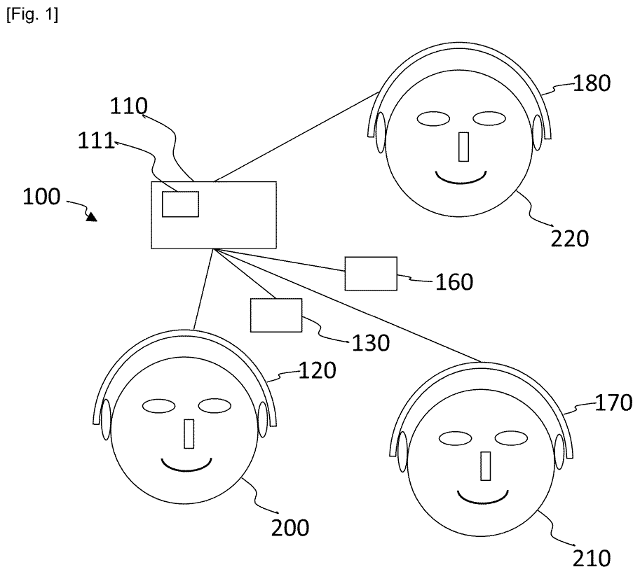

FIG. 1 shows an electronic device according to one embodiment of the invention.

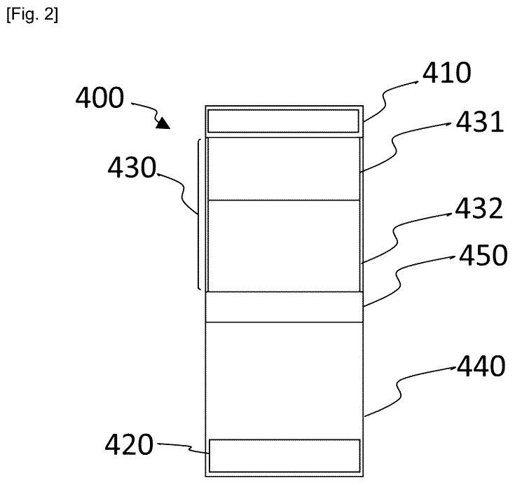

FIG. 2 shows an artificial neural network according to the invention

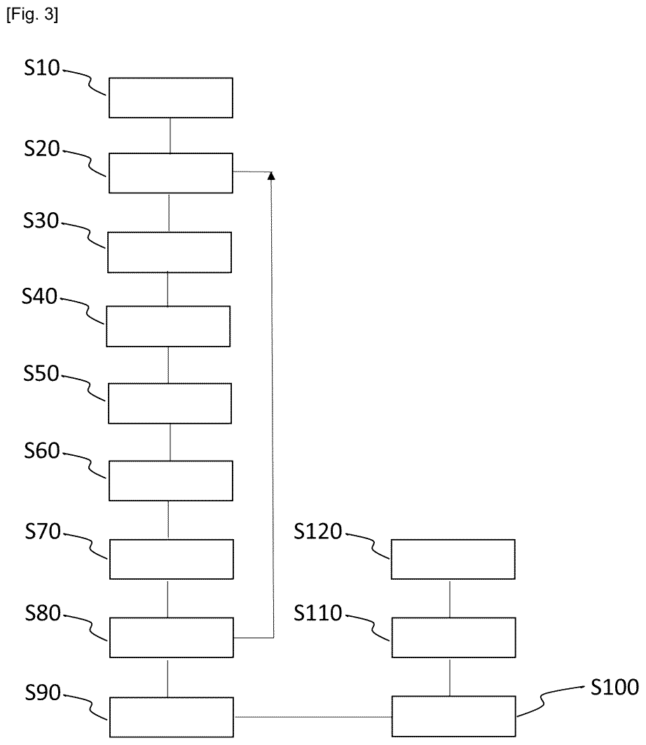

FIG. 3 shows the process according to the invention, in a first embodiment, implemented by the electronic device of FIG. 1.

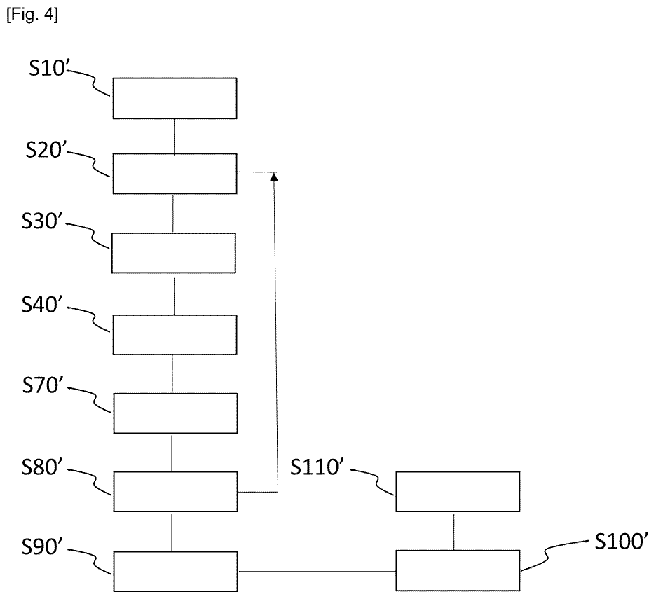

FIG. 4 shows the process according to the invention, in a second embodiment, implemented by the electronic device of FIG. 1.

### DETAILED DESCRIPTION OF AN EMBODIMENT OF THE INVENTION

With reference to FIG. 2, the neural network 400 comprises a first part 430, comprising a first series of layers of the neural network, capable of producing a vector 450 encoding an input data item 410, and a second part 440, comprising at least one layer of neurons, capable of producing the first output character string 420 from the vector 450.

The input data 410 is, for example, an audio file containing the expression (for example in a format known as “wav”, in English for “Waveform Audio File Format” translated into French by the expression format audio de forme d'onde).

For example, the neural network 400 is implemented by the central unit 110.

With reference to FIGS. 1, 2, 3 and 4, in step S10, the first part 430 is pre-trained, prior to the training steps below, from hundreds of hours of input audio files 410, comprising more than 10000 different linguistic expressions (and at most 10000000 of at least first different words).

Preferably, these are any expressions of a natural language.

This is self-supervised training.

Input 410, for example, is truncated (for example, certain parts of the audio file have been removed).

The first part 430 is for example as described in:

Alexei Baevski, Yuhao Zhou, Abdelrahman-Mohamed, Michael Auli: “wav2vec 2.0: A Framework for Self-Supervised Learning of Speech Representations” NeurIPS 2020.

During pre-training, for example, this first part 430 attempts to predict the truncated parts of the input 410 and/or uses a constrative loss to evaluate performance and modify the weights of the neural network (A cost function quantifies the error of the neural network compared to the label and represents the cost as a function of the combinations of parameters of the neural network).

So, following pre-training, the first part 430 is able to encode an input audio file 410 (or audio data) into a vector 450 comprising a representation of the linguistic expressions contained in the audio file.

According to an embodiment, in this first part 430, the weights, of the convolutional encoder 431 (noted f) in the Alexei Baevski et al. paper above which produces a latent representation of the input, are fixed during the training step. The remainder 432 of the first part 430, in particular in the transformer (noted g), in the Alexei Baevski et al. paper above, is modified during training.

The second part 440 can be a single output layer comprising all the phonemes contained in the lists (the output vector indicates which phonemes are known). A “softmax” and a “log” can be applied to the output. This layer can be linear, i.e., with a linear activation function.

In step S20, the electronic device 100 controls the emission of a linguistic expression by a headset 170.

The linguistic expression is, for example, the word “cru”.

The expression may consist of a single word or a single word and an article preceding that word (e.g. “le rondin”) or a phrase.

The expression may comprise a logatome (i.e., a word with no meaning) or be made up of logatomes.

At step S30, the patient 210 responds by reproducing in speech what he has heard.

At step S40, the central unit 110 (which has the architecture of a computer, microprocessor or microcontroller, for example) receives the patient's vocal response via the microphone 130 and records it in memory 111 in the form of an audio file.

For example, patient 210 can say “dru” when pronouncing this word.

In step S50, the recorded response is then listened to by the human operator 220 using the headset 180.

At step S60, the human operator 220 uses the keyboard 160 to enter a string of characters.

At step S70, the character string “dru” is received from the keyboard 160 by the central processing unit 110.

In step S80, the neural network is trained using the recorded response (in the form of an audio file) as input 410 labelled with the character string “dru”.

According to an embodiment, the training steps S20, S30, S40, S50, S60, S70 and S80 are repeated (for example, at least ten thousand times), for different patients, for a series of different expressions in place of the expression “cru”.

The series of different expressions can consist of less than 300 different expressions or less than 2000 different expressions.

The series of different expressions can be made up, for example, of Lafon's cochlear lists comprising 20 lists of 17 words, i.e., 340 expressions, numbered from 1 to 20. Table 1 shows an extract from these lists (more precisely, lists 1 to 6).

For example, steps S20, S30, S40, S50, S60, S70 and S80 can be carried out for 6500 patients:
3500 patients for each word in lists 1 to 5, then
1500 (other) patients for each word in lists 6 to 10, then
2000 (other) patients for each word in lists 11 to 15, then for
2500 (other) patients for each word in lists 16 to 20.

TABLE 1

| List 1 | List 2 | List 3 | List 4 | List 5 | List 6 |
|---|---|---|---|---|---|
| Buée | Bile | Rôde | Abbé | Balle | Bille |
| Ride | Dros sage | Fente | Sud | Soude | Doute |
| Foc | Gaine | Tige | Fausse | Mur | Faine |
| Agis | Fil | Grain | Joute | Nef | Longe |
| Vague | Cru | Cave | Dague | Change | Gave |
| Croc | Boule | Bulle | Acquis | Gage | Seul |
| Lobe | Cale | Somme | Ville | Trou | Ami |
| Mieux | Bonne | Maine | Mare | Mal | Tasse |
| Natte | Rive | Preux | Noce | Tonne | Chene |
| Col | Sol | Bord | Appas | Peur | Pré |
| Fort | Tempe | Rouille | Route | Rampe | Sur |
| Soupe | Fauve | Oser | Cil | Puce | Crin |
| Tonte | Phase | Site | Fete | Cor | Vol |
| Vêle | Mule | Bouée | Veule | Vite | Front |
| Nage | Chatte | Sauve | Chaise | Rance | Ruse |
| Souche | Règne | Chance | Bache | Mouche | Louche |
| Rogne | | Gagne | Souille | Fille | Bagne |
| | | | | bille | |
| | | | | doute | |

Of the 10500 patients, 3000 may be hard of hearing, for example.

For training, the cost function used is, for example, of the connectionist temporal classification type.

In step S90, an audiometric test on a patient 200 is initiated by the electronic device 100. To simplify the description of this embodiment, the pre-training, the training on the patient 210, and the audiometric test on the patient 200 are performed by the same electronic device 100, but in general, most often, these three steps are performed by different devices.

At step S90, the expression “cru” is emitted by the central processing unit 110 using the headphones 120. The expression can be stored in memory 111.

Patient 200 may respond with “dru”, for example.

At step S100, the central unit 110 receives the patient's “dru” voice response via the microphone 130 and records it in the form of an audio file.

In step S110, the neural network 400, implemented by the central unit 110, determines the output 420 of the character string “dru” from the response of the patient 200 in the form of an audio file at the input 410 of the neural network 400.

In step S120, “cru” is compared with “dru” to assess the hearing of patient 200.

Steps S90, S100, S110 and S120 can be repeated for different expressions for which the neural network 400 has been trained. In this way, the patient's hearing 200 is assessed.

For example, steps S90, S100, S110 and S120 can be repeated for the other words in list 2 table 1.

The artificial neural network 400 is, for example, a convolutional network. The first part 430 may comprise a transformer.

FIG. 4 shows a second example of the process according to the invention. In this second example, steps S10′, S20′, S30′, S40′, S90′ and S100′ are identical to steps S10, S20, S30, S40, S90 and S100 respectively.

At step S70′, the character string “F” is received from the keyboard 160 by the central processing unit 110. “F” indicates that the patient 210 has issued an incorrect response, as “cru” is different from “dru”. The character string “V” would indicate that patient 210 has given a correct response. In another example, the character string “FVV” may be received from the keyboard 160 by the central processing unit 110. “F” indicates that the patient 210 has given an incorrect answer and “V” that the following phonemes are correct, because “c” is different from “d”.

In step S80′, the neural network is trained using the recorded response (in the form of an audio file) as input 410 labelled with the character string “F” or “FVV”.

The training steps can be repeated in the same way as for the first example in FIG. 3.

In step S110′, in order to assess the hearing of the patient 200, the neural network 400, implemented by the central unit 110, determines the character string “F” or “FVV” at the output 420 from the response of the patient 200 in the form of an audio file at the input 410 of the neural network 400.

Steps S90′, S100′, S110′ can be repeated for different expressions for which the neural network 400 has been trained. In this way, the patient's hearing 200 is assessed.

1. A method of speech audiometry testing of a first patient comprising the following steps:
first acoustic emission of a recording of a linguistic expression comprising at least one emission phoneme,
first acoustic reception of a first vocal response from the first patient to the first acoustic emission comprising at least one response phoneme,
determination, by an artificial neural network comprising an input and an output, from an input data item obtained from the first response, of a first output character string representative of the at least one response phoneme,
comparison of the first character string with a second character string, representative of said at least one input phoneme, and
prior to the determination step, the artificial neural network is trained by implementing the following training steps:
second acoustic emission of the expression,
second acoustic reception of a second vocal response from a second patient to the second acoustic emission, the second response comprising at least one training phoneme,
reception of a third character string representative of at least one training phoneme, and
supervised training of the artificial neural network from the second input response labelled by the third character string.

2. The speech audiometry test method according to claim 1 in which the expression consists of a single word or a single word and an article preceding that word.

3. The speech audiometry test method according to claim 1 in which the expression consists of more than one word.

4. The speech audiometry test method according to claim 1, in which the neural network comprises a first part, comprising a first series of layers of the neural network, capable of producing a vector encoding the input data, and a second part, comprising at least one layer of neurons, capable of producing the first output character string from the vector.

5. The speech audiometry test method according to claim 4 in which the first part is pre-trained, prior to the training steps, from more than 10000 different words.

6. The method of speech audiometry testing according to claim 4 in which the first part is pre-trained from a truncated input.

7. The speech audiometry test method according to claim 4 in which a portion of the first part has fixed weights during the supervised training step.

8. The speech audiometry test method according to claim 1 in which the data is an audio file comprising the expression.

9. The speech audiometry test method according to claim 1 in which the training steps comprise the following step:
keyboard input of the third string of characters by a human.

10. The method of speech audiometry testing according to claim 1 in which the training steps are repeated for different second patients.

11. The speech audiometry test method according to claim 10 in which all or some of the various second patients are hard of hearing.

12. The speech audiometry test method according to claim 1 in which the training steps are repeated for a series of different expressions.

13. The speech audiometry test method according to claim 12 in which the series of different expressions consists of less than 2000 different expressions.

14. An electronic audiometric test device configured to implement the steps of the method according to claim 1.

15. A computer program embodied on a non-transitory computer readable medium and comprising instructions, executable by a microprocessor or microcontroller, for implementing the method according to claim 1, when the computer program is executed by the microprocessor or microcontroller.

16. The speech audiometry test method according claim 2, in which the neural network comprises a first part, comprising a first series of layers of the neural network, capable of producing a vector encoding the input data, and a second part, comprising at least one layer of neurons, capable of producing the first output character string from the vector.

17. The speech audiometry test method according claim 3, in which the neural network comprises a first part, comprising a first series of layers of the neural network, capable of producing a vector encoding the input data, and a second part, comprising at least one layer of neurons, capable of producing the first output character string from the vector.

18. The method of speech audiometry testing according to claim 5 in which the first part is pre-trained from a truncated input.

19. The speech audiometry test method according to claim 5 in which a portion of the first part has fixed weights during the supervised training step.

20. The speech audiometry test method according to claim 6 in which a portion of the first part has fixed weights during the supervised training step.
